(19) United States
(12) Patent Application Publication
(10) Pub. No.: US 2006/0241393 A1
(43) Pub. Date: Oct. 26, 2006

(54) Rotation positioning device for a coil of a magnetic resonance imaging apparatus

(76) Inventors: Ke Cheng Liu, Zhejiang Hangzhou (CN); Ting Qiang Xue, Shenzhen (CN); Jian Wang, Shenzhen (CN)

Correspondence Address: SCHIFF HARDIN LLP;Patent Department, 6600 Sears Tower, 233 South Wacker Drive, Chicago, IL, 60606, US

(21) Appl. No.: 11/323,668

(22) Filed: Dec. 30, 2005

(30) Foreign Application Priority Data
Dec. 31, 2004 (CN) 200410082380.4

(51) Int. Cl. A61B 5/05

(52) U.S. Cl. 600/422

(57) ABSTRACT

A rotation positioning device for a coil of a magnetic resonance imaging apparatus, having a cavity in the coil for accommodating a part to be examined, has a backing member that can freely rotate within this accommodating cavity. The backing member is provided with a first driving member and a first rotating member, and the coil is provided with a second driving member and a second rotating member. The first driving member and the second driving member form a transmission pair, and the first rotating member can rotate relative to the second rotating member under the drive of the first driving member and the second driving member. The second driving member can be driven manually or by an external driving source connected thereto to drive the first driving member, thereby driving the backing member to rotate via the cooperation of the first rotating member and the second rotating member. Also, by appropriate configuration of parameters, such as position and size, of the first driving member and the second driving member, the rotation angle or range of the backing member can be controlled precisely and quantitatively.

### BACKGROUND OF THE INVENTION

1. Field of the Invention

The present invention relates to a positioning device for a coil of a magnetic resonance imaging (MRI) apparatus, and more particularly to a rotation positioning device for a coil of an MRI apparatus.

2. Description of the Prior Art

In recent years, MRI has become an important tool in diagnostic medicine. MRI employs radio frequency (RF) energy as a stimulus, to observe a magnetic resonance signal emitted by atomic nuclei of a specific type during the process from being perturbed to regaining equilibrium under the effect of a strong static magnetic field. The magnetic resonance signal is converted to an electrical signal using Faraday's law, and an image of the atomic density in an object is calculated via a two dimensional Fourier transform.

Using MRI to perform an examination has the following advantages:

i) among current means of medical imaging, MRI has the highest contrast resolution for soft tissues, and it is capable of clearly distinguishing soft tissues, such as muscle, muscle tendon, organs and fat, and discriminating endocardium of relatively high signals, cardiac muscle of medium signals, epicardium against the background of fat of high signals as well as pericardium of low signal;

ii) MRI is capable of producing an image in an arbitrary direction directly without changing the posture of a patient being examined, and in combination with images in different directions, the structure of an organ or tissue being examined can be displayed from all sides with no dead angle of observation, and a volume scan developed and used in recent years can be used to perform real-time reconstruction of various planes, curved surfaces or irregular sections and to conveniently carry out three-dimensional tracing of anatomical structure and pathological changes;

iii) MRI is a noninvasive and radiation-free examination, avoiding damage caused by radiation in imaging techniques such as x-ray imaging and radionuclide imaging and being harmless to the human body;

iv) MRI has many imaging parameters and includes a large amount of information, and at present, with up to more than ten known MRI imaging parameters plus more than one hundred pulse sequence combinations as well as applications of many special imaging techniques, MRI provides a broad research field for clinical applications; and

v) MRI has a relatively high spatial resolution.

An MRI apparatus basically includes a magnet system, a computer system and an image displaying system. The magnet system includes a main magnet, gradient coil(s), shim coil(s) and RF coil(s) perpendicular to a main magnetic field, forming the main part that causes magnetic resonance to occur and generates signals.

A typical head coil of an MRI apparatus is shown in FIG. 1. The head coil 30 is an open head coil, having a base 32 and a top 34. The base 32 defines a concave semicircular cambered surface thereon. The top 34 is arcuate and an internal surface thereof is designed to be a semicircular cambered surface corresponding to the semicircular cambered surface of the base 32. The top 34 is fixed on top of the base 32, the two corresponding semicircular cambered surfaces forming a cavity 36 for accommodating a body part to be examined such as a head, a cervical spine or a knee. At present, many commercially available MRI apparatus, such as the Signa Openspeed 0.7T™ manufactured by GE Medical Systems, employ a head coil that is identical with or similar to that described above.

Although, as described above, MRI is capable of producing an image in an arbitrary direction directly, the direct production of an image in an arbitrary direction is limited to only some fixed positions, thus an MRI apparatus employing such a head coil has certain disadvantages. The body part to be examined, once fixed by a support bracket, a soft mat, an air bag or a vacuum bag within the accommodating cavity, cannot move quantitatively in any direction. Moreover, even though the body part to be examined can be adjusted properly in terms of position in the direction perpendicular to the accommodating cavity, when the examination needs to produce an image with the body part to be examined in other particular position, such as the position of the body part after it rotates through a particular angle, an MRI apparatus that employs such a head coil cannot meet such a requirement.

Commercially available MRI apparatuses are designed to accommodate different postures by a patient to be examined during an examination, such as the Upright™ MRI apparatus manufactured by FONAR Corporation, which permits the patient to be examined to occupy a supine posture, a sitting posture, a standing posture or other postures to conveniently meet particular requirements when being examined. However, MRI apparatus of this type still does not solve the problem that the body part to be examined of the patient after being fixed, cannot move.

### SUMMARY OF THE INVENTION

An object of the present invention is to provide a rotation positioning device for a coil of an MRI apparatus that allows free rotation of a body part to be examined.

Another object of the present invention is to provide a rotation positioning device for a coil of an MRI apparatus for achieving precise positioning of a body part to be examined.

These objects are achieved in accordance with the present invention by a rotation positioning device for a coil of an MRI apparatus, a cavity being provided in the coil for accommodating a body part to be examined, wherein the rotation positioning device has a backing member that can freely rotate within the accommodating cavity, the backing member being provided with a first driving member and a first rotating member and the coil is provided with a second driving member and a second rotating member. The first driving member and the second driving member form a transmission pair and engage each other, the first rotating member being able to rotate relative to the second rotating member under the drive of the first driving member and the second driving member.

The rotation positioning device according to the present invention further has a locking mechanism for locking the backing member when it rotates to a desired position, and optionally the locking mechanism may be a locking screw or a spring-and-ball self-locking mechanism.

The first driving member and the second driving member can respectively select a proper transmission mode, such as a rack and a gear. The first rotating member and the second rotating member can be a sliding recess and a guiding rail or a sliding pin and a guiding recess, respectively, wherein the sliding recess can be fit over the guiding rail and slide along it, or alternatively the sliding pin can be inserted in the guiding recess and slide along it. The second driving member can be driven manually or by an external driving source connected thereto to drive the first driving member, thereby driving the backing member to rotate through the cooperation of the first rotating member and the second rotating member. Also, by appropriate configuration of parameters, such as position and size, of the first driving member and the second driving member, the rotation angle or range of the backing member can be controlled precisely and quantitatively.

The rotation positioning device for the coil of the MRI apparatus according to the present invention achieves free rotation and precise positioning of the body part to be examined, thus solving the problem that of the body part to be examined, once fixed by a bearing bracket, a soft mat, an air bag or a vacuum bag within the accommodating cavity, cannot move quantitatively in any direction, and meeting the requirement that when the coil of the MRI apparatus needs to perform an examination with the body part to be examined in other particular positions, such as the position of the body part after it rotates through a particular angle.

### DESCRIPTION OF THE DRAWINGS

FIG. 1 is a schematic illustration of a prior art head coil for an MRI apparatus.

FIG. 2 is a schematic illustration of a preferred embodiment of a rotation positioning device for a coil of an MRI apparatus according to the present invention.

FIG. 3 is an enlargement of the portion III of FIG. 2.

FIG. 4 is a schematic illustration of another preferred embodiment of the rotation positioning device for a coil of an MRI apparatus according to the present invention.

FIG. 5 is an enlargement of the portion V of FIG. 4.

FIG. 6 is a schematic illustration of another preferred embodiment of the rotation positioning device for a coil of an MRI apparatus according to the present invention.

FIG. 7 is a schematic illustration of the rotation positioning device for the coil of the MRI apparatus according to the present invention, showing an external drive.

FIG. 8 shows resulting images obtained with the rotation positioning device for the coil of the MRI apparatus according to the present invention.

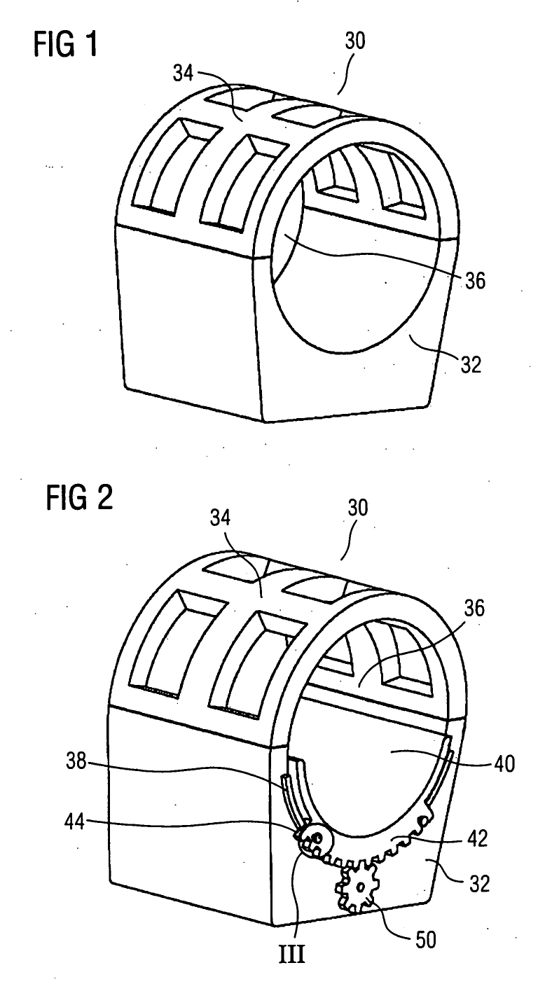

### DESCRIPTION OF THE PREFERRED EMBODIMENTS

A rotation positioning device according to the present invention is provided on a coil of an MRI apparatus, for allowing free rotation and precise positioning of a body part to be examined. The present invention can be widely applied to various coils of the MRI apparatus. Although only one example in which the present invention is applied to a head coil is discussed in the preferred embodiment and succeeding embodiments, the present invention is not limited to this. As will be understood by those skilled in the art, devices applied to other coils that are identical with or similar to the rotation positioning device for the coil of the MRI apparatus according to the present invention fall within the scope of the present invention.

Referring to FIG. 2, a head coil 30 has a base 32 and a top 34 that is fixed on top of the base 32. It is understood that the base 32 and the top 34 may also be integrated with each other. An upper surface of the base 32 defines a concave semicircular cambered surface, and the top 34 is arc in shape, also defining a semicircular cambered surface corresponding to the semicircular cambered surface of the base 32. When the top 34 is fixed to the base 32, the two corresponding semicircular cambered surfaces form an accommodating cavity 36 therebetween for accommodating a body part to be examined, such as a head, a cervical spine or a knee.

An arc-shaped backing member 40 is arranged within the accommodating cavity 36 and can rotate freely along an internal wall of the accommodating cavity 36. One end face of the backing member 40 extends outwardly for a proper distance along the edge thereof and forms a first driving member 42. In this preferred embodiment, the first driving member 42 may be a rack. The first driving member 42 can be integrated with the backing member 40 as described above or a separate element secured at an outer side of an end of the backing member 40 by a securing element such as a screw. A second driving member 50 is mounted on an end face of the base 32 of the head coil 30 at the same side as the end face of the backing member 40 and engages with the first driving member 42 to drive it. In this preferred embodiment, the second driving member 50 may be a gear. The radius, number of teeth and teeth space of the rack and said gear can be configured appropriately depending on their applications to different coils. A first rotating member 44 is provided on the first driving member 42. In this preferred embodiment, the first rotating member 44 may be an arc-shaped sliding trough which extends through the first driving member 42 along an external wall of the backing member 40. A second rotating member 38 is provided on the end face of the base 32 of the head coil 30. Similarly, the second rotating member 38 may also be integrated with or secured to the end face of the base 32. In this preferred embodiment, the second rotating member 38 may be a guiding rail matching the shape of the first rotating member 44 so that the sliding trough can be fit over the guiding rail and slide along it. The first rotating member 44 and the second rotating member 38 rotate cooperatively so that the backing member 40 rotates along the internal wall of the accommodating cavity 36. The first driving member 42 and the second driving member 50 cooperate to drive the first rotating member 44 and the second rotating member 38 and precisely control the rotation angle or range of the first rotating member 44 relative to the second rotating member 38 by appropriate configuration of, for example, the radius, number of teeth and teeth space.

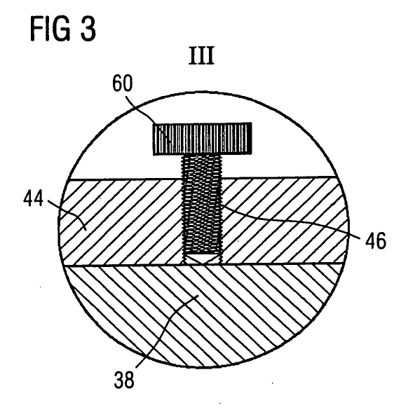

The present invention further has a locking mechanism for locking the first rotating member 44 when it rotates to a desired position relative to the second rotating member 38. Also, referring to FIG. 3, the locking mechanism may be a locking screw 60. There is provided a locking bore 46 in the first rotating member 44, and the locking screw 60 extends through the locking bore 46 and abuts against the second rotating member 38, thereby securing the first rotating member 44 to the second rotating member 38.

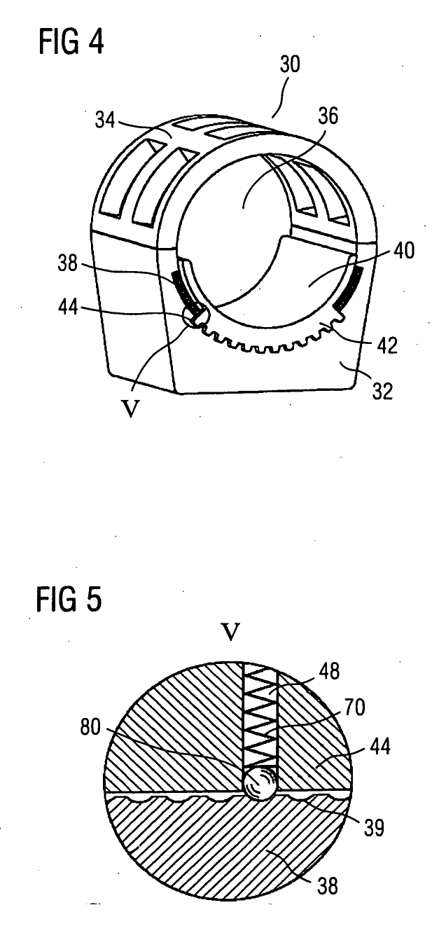

Also, referring to FIGS. 4 and 5, in another preferred embodiment of the present invention, the locking mechanism may also be a spring-and-ball self-locking mechanism. As shown in the Figures, there is provided an accommodating cavity 48 in the first rotating member 44, and a spring 70 and a ball 80 are mounted in turn in the accommodating cavity 48. The second rotating member 38 defines several positioning recesses 39 on the surface thereof, and the ball 80 can be positioned in one of the several recesses 39 under the elastic force of the spring 70, thereby securing the first rotating member 44 to the second rotating member 38. As the first rotating member 44 is driven, the ball 80 can slide along the surface of said several recesses 39, and the ball 80, when sliding to a desired position, will no longer be driven and can be positioned at a corresponding positioning recess 39. The radius of the ball 80 and the spaces between the positioning recesses 39 can be configured appropriately depending on applications to different coils.

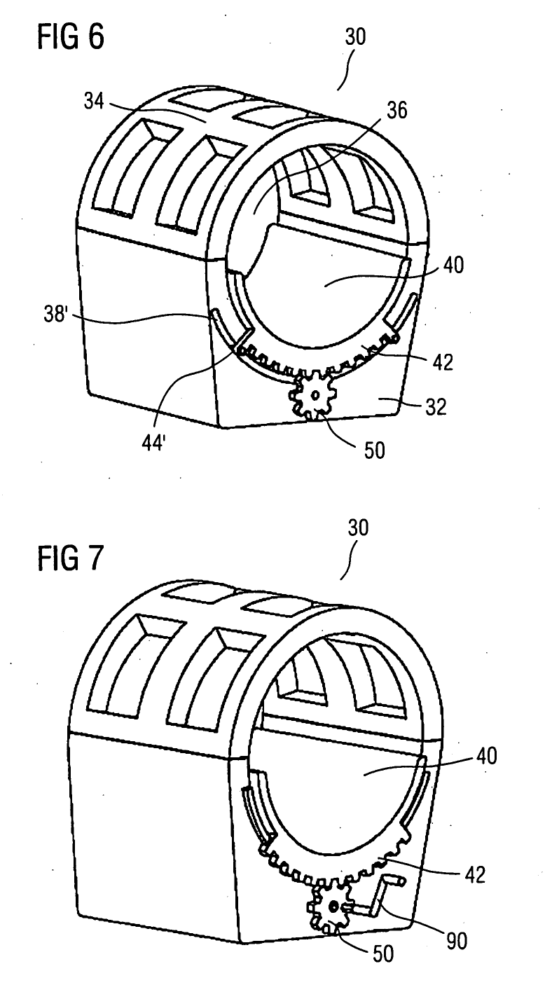

Referring to FIG. 6, in another preferred embodiment of the present invention, the first rotating member may also be a sliding pin 44′. The sliding pin 44′ is fixed to the first driving member 42. The second rotating member may be an arc-shaped sliding trough 38′ correspondingly. The sliding pin 44′ is inserted in the guiding trough 38′ and can slide freely therein, thereby causing the backing member 40 to rotate along the internal wall of the accommodating cavity 36. The first driving member 42 and the second driving member 50 cooperate to drive the sliding pin 44′ to slide in the guiding trough 38′, and precisely control the rotation angle or range of the sliding pin 44′ relative to the guiding trough 38′ by appropriate configuration of, for example, the radius, number of teeth and teeth space of the first driving member 42 and the second driving member 50. When the sliding pin 44′ slides to a desired position, the backing member 40 can be locked by the locking mechanism.

An external drive for the first driving member 42 and the second driving member 50 may be manual or be selected from a pneumatic motor, a hydraulic motor, a winding mechanism and so on. Referring to FIG. 7, in this preferred embodiment, the external drive can drive the first driving member 42 and the second driving member 50 by a manual cranking bar 90. The manual cranking bar 90 is provided on the second driving member 50, and by turning the manual cranking bar 90 the second driving member 50 can be driven to drive the first driving member 42, whereby the backing member 40 is driven to rotate via the cooperation of the first rotating member 44 and the second rotating member 38.

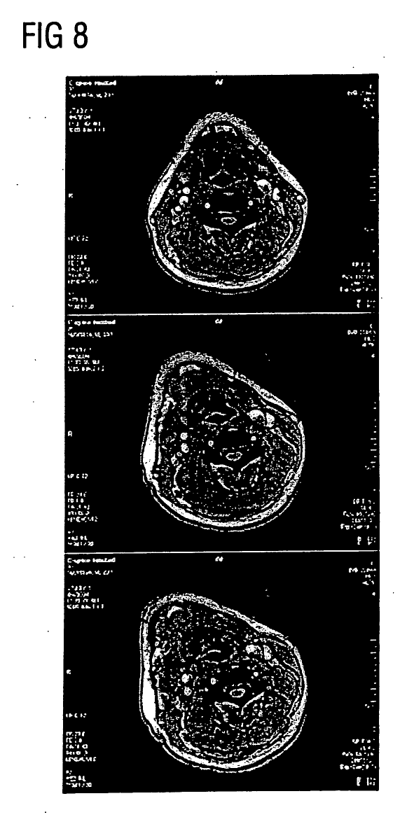

When the MRI apparatus employing the present invention is used to perform an examination, the part to be examined can be disposed within the accommodating cavity 36 with the backing member 40 put underneath, and then the part to be examined can be fixed by a bearing bracket, a soft mat, an air bag or a vacuum bag. If the part to be examined needs to be rotated through a proper angle during the examination, the external drive is operated, and, by quantitative drive control of the first driving member 42 and the second driving member 50 and the resulting cooperation of the first rotating member 44 and the second rotating member 38, the backing member 40 rotates to a desired position, thereby rotating the body part to be examined to a desired position, and finally the backing member 40 is locked by said locking mechanism. Therefore, with the present invention, free rotation and precise positioning of the body part to be examined can be achieved, and resulting images obtained from different angles when the MRI apparatus employing the present invention is used to examine the body part to be examined are shown in FIG. 8.

Although modifications and changes may be suggested by those skilled in the art, it is the intention of the inventors to embody within the patent warranted hereon all changes and modifications as reasonably and properly come within the scope of their contribution to the art.

We claim as our invention:

1. A rotation positioning device for a magnetic resonance imaging coil, comprising:
a magnetic resonance imaging coil having a cavity adapted to receive an examination subject;
a backing member that is freely rotatable within said cavity;
a first driving member and a first rotating member carried by said backing member;
a second driving member and a second rotating member attached to said coil; and
said first driving member and said second driving member forming a transmission pair, and said first rotating member being rotated relative to said second rotating member upon being driven by said transmission pair.

2. A rotation positioning device as claimed in claim 1 comprising a locking mechanism that locks said backing member after said backing member has rotated to a selected position.

3. A rotation positioning device as claimed in claim 1 wherein said coil is a head coil.

4. A rotation positioning device as claimed in claim 1 wherein said backing member has an arcuate shape adapted to support said examination subject.

5. A rotation positioning device as claimed in claim 1 wherein said first driving member is a rack.

6. A rotation positioning device as claimed in claim 5 wherein said second driving member is a gear.

7. A rotation positioning device as claimed in claim 1 wherein said first rotating member is a sliding groove.

8. A rotation positioning device as claimed in claim 7 wherein said sliding groove is disposed along said first driving member.

9. A rotation positioning device as claimed in claim 7 wherein said second rotating member is a guiding rail.

10. A rotation positioning device as claimed in claim 9 wherein said sliding groove fits over said guiding rail and slides along said guiding rail.

11. A rotation positioning device as claimed in claim 1 wherein said first rotating member is a sliding pin.

12. A rotation positioning device as claimed in claim 11 wherein said sliding pin is attached to said first driving member.

13. A rotation positioning device as claimed in claim 11 wherein said second rotating member is a guiding groove.

14. A rotation positioning device as claimed in claim 13 wherein said sliding pin is disposed in said guiding groove and slides therein.

15. A rotation positioning device as claimed in claim 1 comprising a locking mechanism that locks said backing member after said backing member has rotated to a selected position, said locking mechanism comprising a locking screw that extends through a locking bore in said first rotating member and abuts said second rotating member.

16. A rotation positioning device as claimed in claim 1 comprising a locking mechanism that locks said backing member after said backing member has rotated to a selected position, said locking mechanism comprising a spring-and-ball self-locking mechanism.

17. A rotation positioning device as claimed in claim 16 wherein said spring-and-ball self-locking mechanism comprises a cavity in said first rotating member, a spring and a ball mounted successively in said cavity in said first rotating member, and a plurality of positioning recesses defined in a surface in said second rotating member, said ball being positionable in one of said positioning recesses by a force exerted by said spring.

18. A rotation positioning device as claimed in claim 1 wherein said second driving member has a manually operable crank that, when manually operated, causes said second driving member to drive said first driving member.

19. A rotation positioning device as claimed in claim 1 comprising an external driving source connected to said second driving member, said external driving source driving said second driving member to drive said first driving member.

20. A rotation positioning device as claimed in claim 1 wherein said external driving source is a driving source selected from the group consisting of pneumatic motors, hydraulic motors, and winding mechanisms.
